(19) United States
(12) Patent Application Publication
(10) Pub. No.: US 2010/0196514 A1
(43) Pub. Date: Aug. 5, 2010

(54) CALCIUM SILICATE-BASED COMPOSITE CEMENT AND METHODS FOR THE PREPARATION

(75) Inventor: Shinn-Jyh Ding, Taichung City (TW)

Correspondence Address: WPAT, PC;INTELLECTUAL PROPERTY ATTORNEYS, 2030 MAIN STREET, SUITE 1300, IRVINE, CA, 92614, US

(73) Assignee: Chung Shan Medical University, Taichung City (TW)

(21) Appl. No.: 12/364,278

(22) Filed: Feb. 2, 2009

(51) Int. Cl. A61K 33/08; A61P 19/08

(52) U.S. Cl. 424/682

(57) ABSTRACT

The present invention provides a method for producing calcium silicate-based bone cement and a composition produced by the method. It further provides a novel composition for bone tissue repair.

### FIELD OF THE INVENTION

This present invention relates to bone cement especially calcium silicate-based bone cements containing polymers and/or oligomers.

### BACKGROUND

Silicon (Si), an important trace element in the early stages of bone formation, increased directly with calcium at relatively low calcium concentrations and then fell below the detection limit at compositions approaching hydroxyapatite (Carlisle E M, Silicon: a possible factor in bone calcification. Science 1970;167:279-280.). The soluble form of silicon may stimulate collagen type I synthesis and osteoblastic differentiation in human osteoblast-like cells (Reffitt D M, Ogston N, Jygdaohsingh R. Orthosilicic acid stimulates collagen type I synthesis and osteoblastic differentiation in human osteoblast-like cells in vitro. Bone 2003;32:127-135.). Silicate-based materials may be promising for use in the reconstruction of frontal sinus and spine, augmentation of craniofacial skeletal defects and osteoporosis, endodontics, and repair of periodontal bone defects. Calcium silicate-based ceramics such as CaSiO₃ (Siriphannon P, Kameshima Y, Yasumori A, Okada K, Hayashi S. Influence of preparation conditions on the microstructure and bioactivity of α-CaSiO₃ ceramics: formation of hydroxyapatite in simulated body fluid. J Biomed Mater Res 2000;52:30-39; Sarmento C, Luklinska Z B, Brown L, Anseau M, De Aza P N, De Aza S. In vitro behavior of osteoblastic cells cultured in the presence of pseudowollastonite ceramic. J Biomed Mater Res 2004;69A:351-358.), bioactive glass (Saravanapavan P, Jones J R, Pryce R S, Hench L L. Bioactivity of gel-glass powders in the CaO—SiO₂ system: A comparison with ternary (CaO—P₂O₅—SiO₂) and quaternary glasses (SiO₂—CaO—P₂O₅—Na₂O). J Biomed Mater Res 2003;66A:110-119.), and mineral trioxide aggregate (Ribeiro D A, Duarte M A H, Matsumoto M A, Marques M E A, Salvadori D M F. Biocompatibility in vitro tests of mineral trioxide aggregate and regular and white Portland cements. J Endod 2005;31:605-607.) were good bioactive materials for bone defect repair in orthopedic and dental surgery because of their excellent bioactivity.

Sol-gel derived calcium silicate materials have been studied for the use of bulk or scaffold. Izquierdo-Barba et al. synthesized a bioactive glass with a composition of 80SiO₂/2OCaO (in mol %) by sol-gel method (Izquierdo-Barba I, Salinas A J, Vallet-Regi M. In vitro calcium phosphate layer formation on sol-gel glasses of the CaO—SiO₂ system. J Biomed Mater Res 1999;47:243-250.). The same research group also found that the rate of apatite formation on the SiO₂—CaO glass with lower SiO₂ (50-70% in mol) is greater than those glasses with higher SiO₂ (80-90% in mol) when soaked in a simulated body fluid (Martinez A, Izquierdo-Barba I, Vallet-Regi M. Bioactivity of a CaO—SiO₂ binary glasses system. Chem Mater 2000;12:3080-3088.).

Self-setting cements can be handled by the surgeon in paste form and injected into bone cavities or defects, and set to form a mineral matrix. The setting time is one of most clinically relevant factors. A long setting duration could cause clinical problems due to the cement's inability to maintain shape and support stresses during this time period (Ishikawa K, Miyamoto Y, Takechi M, Toh T, Kon M, Nagayama M, Asaoka K. Non-decay type fast-setting calcium phosphate cement:hydroxyapatite putty containing an increased amount of sodium alginate. J Biomed Mater Res 1997;36:393-399.). Particle size, sintering temperature, liquid phase, and composition of powders, as well as the ratio of liquid to powder, play a crucial role on the setting time of paste materials. On the other hand, the prerequisite for materials to bond to living bone is the formation of a “bone-like” apatite layer, an indicator of bioactivity (the ability to form a chemical bond with living tissue), on their surface in the body. The cement must promote the precipitation of a “bone-like” HA layer on the cement surfaces when exposed to a physiological solution, which may support its ability to integrate with living tissue.

As for the sol-gel derived calcium silicate cements, Chang and his co-workers prepared dicalcium silicate and tricalcium silicate powders using sol-gel methods. With the two calcium silicate powders, they can be mixed with water to produce a calcium silicate cement with the initial setting time of higher than 1 hour, on which the apatite precipitation needed to take several days in simulated body fluid (Zhao W, Wang J, Zhai W, Wang Z, Chang J. The self-setting properties and in vitro bioactivity of tricalcium silicate. Biomaterials 2005;26:6113-6121; Gou Z, Chang J, Zhai W, Wang J. Study on the self-setting property and the in vitro bioactivity of β-Ca₂SiO₄. J Biomed Mater Res 2005;73B:244-251.). Very recently we proposed preparation of calcium silicate cements consisting of the sol-gel-derived calcium silicate powder as a solid phase and ammonium phosphate solution as a liquid phase. The cements obtained showed not only fast-setting within 9 minutes, as well as highly bioactive, but also enhanced cell proliferation and differentiation (Ding S J, Shie M Y, Wang C Y. Novel fast-setting calcium silicate bone cements with high bioactivity and enhanced osteogenesis in vitro. J Mater Chem. DOI: 10.1039/B819033J, 2009, in press). However, it is possible to difficultly deliver to the required site and hard to compact adequately due to relatively poor brittleness resistance of a ceramic-based cement. The polymeric materials such as chitosan, alginate, and gelatin should have the potential to improve the handling properties of calcium-silicate cements because of their inherent elasticity.

Bone and teeth are composite materials consisting mainly of an organic matrix (collagen) and a mineral phase (hydroxyapatite). The successful design of bone substitute materials requires an appreciation of bone structure. Thus, the use of a hybrid composite comprised biopolymer and calcium silicate, resembling the morphology and properties of natural bone, may be one way to solve the problem of ceramics' brittleness without reducing mechanical properties, in addition to possessing good biocompatibility, high bioactivity and great bonding properties.

Chitosan is an abundant, naturally occurring polysaccharide obtained by deacetylation of natural chitin with one of the polysaccharides having a free amino group at the C2 position of the glucose residue of cellulose (Francis Suh J K, Matthew H W T. Application of chitosan-based polysaccharide biomaterials in cartilage tissue engineering: a review. Biomaterials 2000;21:2589-2598.). Due to its numerous desirable properties, e.g., low cost, no antigenicity, chemical inertness, low toxicity, high hydrophilicity, and good film-forming property (Denkbas E B, Odabasi M. Chitosan microspheres and sponges: preparation and characterization. J Appl Polym Sci 2000;76:1637-1643; VandeVord P J, Matthew H W T, DeSilva S P, Mayton L, Wu B, Wooley P H. Evaluation of the biocompatibility of a chitosan scaffold in mice. J Biomed Mater Res 2002;59:585-590.), chitosan also became a natural choice for biomedical applications such as microspheres, membranes, and scaffolds. Liu and colleagues developed an injectable bone substitute material consisting of chitosan, citric acid and glucose solution as the liquid phase, and tricalcium phosphate powder as the solid phase (Liu H, Li H, Cheng W, Yang Y, Zhu M, Zhou C. Novel injectable calcium phosphate/chitosan composites for bone substitute materials. Acta Biomater 2006;2:557-565.). This material was moldable because of its paste consistency after mixing. Yokoyama et al. developed a chitosan-containing calcium phosphate cement that could be molded into any desired shape due to its chewing-gum-like consistency (Yokoyama A, Yamamoto S, Kawasaki T, Kohgo T, Nakasu M. Development of calcium phosphate cement using chitosan and citric acid for bone substitute materials. Biomaterials 2002;23:1091-1101.). Xu and Simon developed strong and macroporous CPC scaffolds by incorporating chitosan and water-soluble mannitol, and to examine the biocompatibility of the new graft with an osteoblast cell line and an enzymatic assay (Hockin H. K. Xu, Carl G. Simon Jr. Fast setting calcium phosphate-chitosan scaffold: mechanical properties and biocompatibility. Biomaterials 2005;26: 1337-1348.).

Gelatin is a nature polymer, which is obtained from the bovine bone by thermal denaturation or physical and chemical degradation of collagen. Gelatin has been widely employed as a scaffold material in the field of the tissue engineering or the drug carrier because of biocompatibility, biodegradability and nontoxicity (Olsen D, Yang C, Bodo M, Chang R, Leigh S, Baez J, Carmichael D, Perälä M, Hämäläinen E R, Jarvinen M, Polarek J. Recombinant collagen and gelatin for drug delivery. Adv Drug Delivery Rev 2003;55:1547-1567; Tabata Y, Hong L, Miyamoto S, Miyao M, Hashimoto N, Ikada Y. Bone formation at a rabbit skull defect by autologous bone marrow cells combined with gelatin microspheres containing TGF-β1. J Biomater Sci Polym Edn 2000;11:891-901.). The advantages of gelatin are the usability with different charges and the easiness of chemical modification. Fujishiro and coworkers found that addition of gelatin gel to α-tricalcium phosphate cement resulted in the formation of a porous solid possessing pores of 20-100 μm in diameter whose pore diameter increased with increasing gelatin gel content (Fujishiro Y, Takahashi K, Sato T. Preparation and compressive strength of α-tricalcium phosphate/gelatin gel composite cement. J Biomed Mater Res 2001;54:525-530.). The compressive strength of α-tricalcium phosphate cement after 1 week increased from 9.0 to 14.1 MPa with increasing gelatin gel content up to 5 wt % and thereafter decreased. The use of chitosan fiber and gelatin is to reinforce the mechanical property of CPC (Pan Z, Jiang P, Fan Q, Ma B, Cai H. Mechanical and biocompatible influences of chitosan fiber and gelatin on calcium phosphate cement. J Biomed Mater Res 2007;82B:246-252.). Gelatin at the mass fraction of 5% and chitosan fiber at the volume fraction of 30% are optimal for this reinforcement mode.

### SUMMARY OF THE INVENTION

This present invention provides a method for producing calcium silicate-based bone cement, comprising (a) mixing calcium salt with silicon compound; (b) treating the mixture of step (a) with a sol-gel process; (c) heating the mixture of step (b); (d) adding materials for enhancing plasticity to the mixture of step (c); (e) grinding the mixture of step (d) into powder; and (f) adding the powder to water or phosphate solution with or without the materials for enhancing plasticity, wherein the materials for enhancing plasticity are selected from the group consisting of the polymeric and oligomeric materials with the functional group —NH₂, —OH, —CO or —CH₃.

The present invention further provides a composition for polymer-containing calcium silicate bone cement, comprising (a) calcium salt; (b) silicon compound; (c) materials for enhancing plasticity; and (d) pharmacological acceptable solution with or without the materials for enhancing plasticity, wherein the materials for enhancing plasticity are selected from the group consisting of the polymeric and oligomeric materials with the functional group —NH₂, —OH, —CO or —CH₃.

### BRIEF DESCRIPTION OF THE DRAWINGS

The following detailed description, given by way of examples and not intended to limit the invention to the embodiments described herein, will best be understood in conjunction with the accompanying drawings, in which:

FIG. 1 illustrates XRD patterns for (A) S60C40, (B) S50C50, (C) S40C60, and (D) S30C70 powders after sintering at different temperature.

FIG. 2 illustrates XRD patterns for the five cements which powders were prepared by sintering at 800° C. and the liquid phase was water.

FIG. 3 illustrates XRD patterns for the five calcium silicate-based cements containing (A) gelatin or (B) chitosan.

FIG. 4 illustrates DTS values of the five calcium silicate-based cements containing different amounts of gelatin (GLT). Water and 5 wt % chitosan (CTS) solution were used as the liquid phase to prepare the cement specimens.

FIG. 5 illustrates setting time values of the five calcium silicate-based cements containing with and without 5 wt % gelatin (GLT). Water and 5 wt % chitosan (CTS) solution were used as the liquid phase to prepare the cement specimens.

FIG. 6 illustrates the injectability of the S50C50 cements with and without 5 wt % gelatin (GLT). Water and 5 wt % chitosan (CTS) solution were used as the liquid phase to prepare the cement specimens.

FIG. 7 illustrates DTS values of (A) S60C40, (B) S50C50, (C) S40C60, and (D) S30C70 cement specimens with and without 5 wt % gelatin (GLT) before and after immersion in simulated body fluid for predetermined periods of time. Water and 5 wt % chitosan (CTS) solution were used as the liquid phase to prepare the cement specimens.

FIG. 8 illustrates surface SEM micrographs of (A) S60C40, (B) S50C50, (C) S40C60, and (D) S30C70 cement specimens after immersion for 1 day in simulated body fluid. Water was used as a liquid phase to prepare the cement specimens.

FIG. 9 illustrates surface SEM micrographs of (A) hardened S50C50 cement specimens containing (B) gelatin, (C) chitosan, and (D) gelatin and chitosan after immersion for 1 hour in simulated body fluid. Water and 5 wt % chitosan solution were used as the liquid phase to prepare the cement specimens. The arrows indicate the apatite precipitates.

FIG. 10 illustrates WST-1 assay for attachment and proliferation of human mesenchymal stem cells cultured on the S50C50 cements with and without 5 wt % gelatin (GLT) at various incubation periods. Water and 5 wt % chitosan (CTS) solution were used as the liquid phase to prepare the cement specimens.

FIG. 11 illustrates SEM micrographs of human mesenchymal stem cells cultured on (A) the S50C50 cements containing (B) gelatin, (C) chitosan, and (D) gelatin and chitosan for 1 day. Water and 5 wt % chitosan solution were used as the liquid phase to prepare the cement specimens.

### DETAILED DESCRIPTION OF THE INVENTION

The present invention provides a method for producing calcium silicate-based bone cement, comprising the following steps:

(a) mixing calcium salt with silicon compound;

(b) treating the mixture of step (a) with a sol-gel process;

(c) heating the mixture of step (b);

(d) adding materials for enhancing plasticity to the mixture of step (c);

(e) grinding the mixture of step (d) into powder; and

(f) adding the powder to water or phosphate solution with or without the materials for enhancing plasticity,

wherein the materials for enhancing plasticity are selected from the group consisting of the polymeric and oligomeric materials with the functional group —NH₂, —OH, —CO or —CH₃.

In a preferred embodiment, the materials for enhancing plasticity are selected from the group consisting of the polymeric and oligomeric materials with the functional group —NH₂, —OH, —CO or —CH₃.

In a preferred embodiment, the polymeric and oligomeric materials are selected from the group consisting of gelatin, collagen, chitosan, chitin, cellulose, alginate, hyaluronic acid, poly(lactic acid), poly(glycolic acid), and poly(lactic-co-glycolic acid). The materials for enhancing plasticity as solid phase or liquid phase are weighed in the range of 1 to 50 wt %. Preferably, the amount of materials for enhancing plasticity is 2-30 wt %.

In a preferred embodiment, the calcium salt is calcium nitrate and the silicon compound is silicohydride which of said method has the following formula:

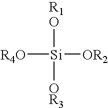

wherein R₁, R₂, R₃ or R₄ is C₁₋₆ alkyl.

In a more preferred embodiment, the silicohyride of said method has the following formula:

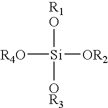

wherein R₁, R₂, R₃ or R₄ is C₂H₅.

In a preferred embodiment, said mixture has a molar ratio of calcium to silicon ranges from 10 to 0.1. In a more preferred embodiment, the mixture has the molar ratio of calcium to silicon range from 4 to 0.25.

The sol-gel process of said method comprises the following steps:

(a) mixing the mixture calcium and silicon precursors with a diluted solvent selected from the group consisting of ethanol and nitric acid for 1˜12 hours;

(b) placing the mixture at the temperature of 20˜80° C. for 1˜7 days; and

(c) drying the mixture at −40˜150° C.

The heating process of said method comprises the following steps:

(a) heating the dried mixture to 700˜1300° C. at the rate of 1˜40° C./min;

(b) maintaining the mixture at a constant temperature ranges from 700˜1300° C.; and

(c) cooling the mixture to room temperature by air-cooling, water-cooling or fast cooling techniques to obtain calcium silicate powder.

The adding process comprises the following steps:

(a) adding the materials for enhancing plasticity to the calcium silicate powder; and

(b) mixing the powder using a conditioning mixer for 5˜30 min.

The grinding process comprises the following steps:

(a) mixing the calcium silicate powder containing the materials for enhancing plasticity with alcohol;

(b) grinding the powder with a ball miller for 0.5˜3 days; and

(c) drying the powder at −40˜60° C.

In a preferred embodiment, the particle size of the powder ranges from 0.01 to 50 micrometers;

In a preferred embodiment, the powder is added to water for 10˜60 seconds and the powder to water is at a ratio of 1 g/0.3-2 mL. In a more preferred embodiment, the powder to water is at a ratio of 1 g/0.4-0.8 mL.

In a preferred embodiment, the powder is added to phosphate solution for 10˜60 seconds and the powder to phosphate solution is at a ratio of 1 g/0.3-2 mL. In the best embodiment, the powder to phosphate solution is at a ratio of 1 g/0.4-0.8 mL.

In a preferred embodiment, the phosphate solution contains an anion selected from phosphate radical (PO₄³⁻), monohydric phosphate radical (HPO₄²⁻) or dihydric phosphate radical (H₂PO₄⁻) with a concentration of 0.12˜5 M, and the phosphate solution contains a cation selected from ammonium, or a member of group IA.

Preferably, the cation of the phosphate solution is ammonium, sodium or potassium.

The present invention further provides a composition for polymer-containing calcium silicate bone cement, comprising

(a) calcium salt;

(b) silicon compound;

(c) materials for enhancing plasticity; and

(d) pharmacological acceptable solution with or without the materials for enhancing plasticity,

wherein the materials for enhancing plasticity are selected from the group consisting of the polymeric and oligomeric materials with the functional group —NH₂, —OH, —CO or —CH₃.

The pharmacological acceptable solution is water, sodium chloride solution, calcium chloride solution or phosphate solution.

Furthermore, the composition can be applied to orthopedic surgery, spine fusion surgery or dental application. It can also serve as replacement bone or tooth material. When applied to human, it can further comprise an excipient for oral administration.

### EXAMPLE

The example below is non-limiting and is merely representative of various aspects and features of the present invention.

### Example 1

Phase Composition of Calcium Silicate Powders

Tetraethyl orthosilicate (Si(OC₂H₅)₄, TEOS) and calcium nitrate (Ca(NO₃)₂.4H₂O) were used as precursors for SiO₂ and CaO, respectively, and nitric acid as catalyst. Ethanol was used as the solvent. The molar ratio of SiO₂/CaO is in the range of 7/3 to 3/7, as listed in Table 1. Various calcium silicate powders were synthesized by sol-gel method. The general procedure of sol-gel route, such as hydrolysis and ageing, was adopted. Briefly, TEOS was hydrolyzed with the sequential addition of 2N HNO₃ and absolute ethanol for 1 hour stirring separately. The required amount of Ca(NO₃)₂.4H₂O was added to the above ethanol solution, the mixed solutions were stirred for an another 1 hour. The molar ratio of (HNO₃+H₂O):TEOS:ethanol was 10:1:10. The sol solution was sealed and aged at 60° C. for 1 day.

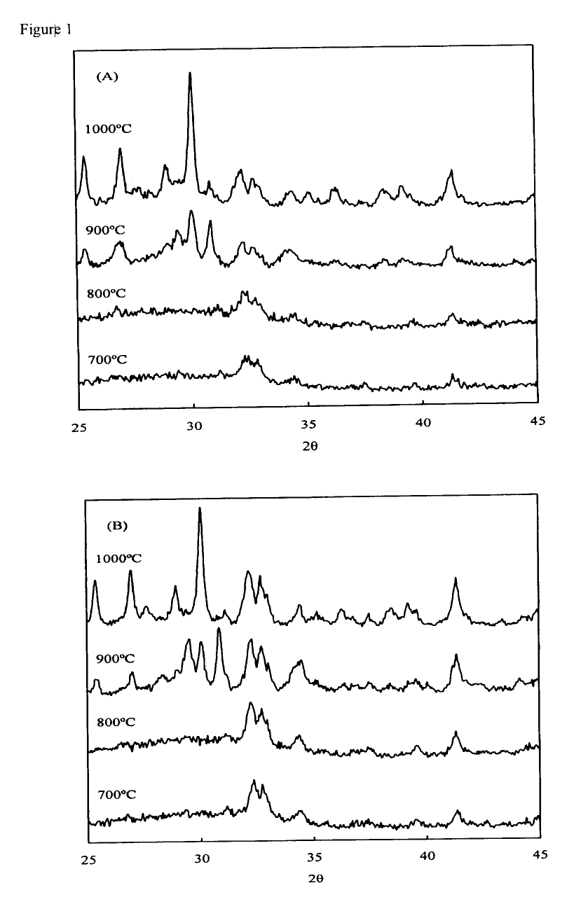

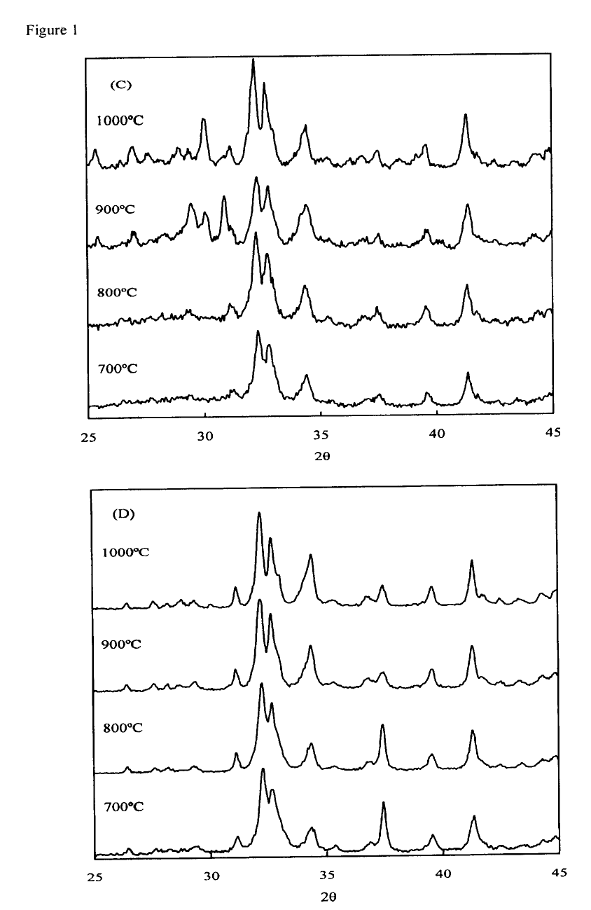

After solvent vaporization of the above-mentioned mixture solution in an oven at 120° C., the as-dried gel was heated in air to 700, 800, 900, or 1000° C. for holding 2 hours, and then cooled to room temperature to produce various calcium silicate powders. Phase analysis was performed using Shimadzu XD-D1 X-ray diffractometer (XRD) with Ni-filtered Cukα radiation operated at 30 kV and 30 mA at a scanning speed of 1°/min. XRD spectra of various as-prepared powders at different sintering temperature are presented in FIG. 1. The diffraction maximum between 29 and 35° at 2θ can be attributed to different crystalline phases of calcium silicates, such as wollastonite and dicalcium silicate. The crystallinity of the powders increased with the increasing sintering temperature.

TABLE 1
Composition (molar ratio), setting time and diametral tensile strength (DTS)
of the five SiO₂—CaO cements prepared by using powders at
different sintering temperature after mixing with water or 0.5 M Na₂HPO₄

| | | Sintering | Water | | 0.5 M Na₂HPO₄ | |
|---|---|---|---|---|---|---|
| Sample | Composition | temperature | Setting time | DTS | Setting time | DTS |
| Code | SiO₂:CaO | (° C.) | (min) | (MPa) | (min) | (MPa) |
| S70C30 | 7:3 | 700 | 38.4 ± 1.9 | 0.1 ± 0.0 | 35.4 ± 1.7 | 0.2 ± 0.0 |
| | | 800 | 42.6 ± 2.1 | 0.6 ± 0.1 | 36.4 ± 2.5 | 0.3 ± 0.1 |
| | | 900 | 44.1 ± 3.0 | 0.4 ± 0.1 | 38.5 ± 1.6 | 0.4 ± 0.1 |
| | | 1000 | 46.0 ± 2.6 | 0.3 ± 0.1 | 41.0 ± 2.1 | 0.5 ± 0.1 |
| S60C40 | 6:4 | 700 | 33.5 ± 1.6 | 0.2 ± 0.0 | 32.8 ± 1.7 | 0.2 ± 0.0 |
| | | 800 | 33.9 ± 2.3 | 2.2 ± 0.3 | 31.5 ± 2.6 | 2.0 ± 0.3 |
| | | 900 | 34.1 ± 1.5 | 1.6 ± 0.3 | 31.9 ± 1.5 | 2.4 ± 0.3 |
| | | 1000 | 35.6 ± 2.1 | 2.3 ± 0.3 | 33.3 ± 2.2 | 2.7 ± 0.4 |
| S50C50 | 5:5 | 700 | 24.8 ± 1.5 | 1.8 ± 0.3 | 24.0 ± 1.9 | 1.7 ± 0.3 |
| | | 800 | 28.3 ± 2.6 | 2.8 ± 0.4 | 25.6 ± 1.4 | 2.8 ± 0.4 |
| | | 900 | 28.5 ± 2.1 | 2.4 ± 0.2 | 27.4 ± 1.7 | 2.8 ± 0.3 |
| | | 1000 | 31.1 ± 1.8 | 2.1 ± 0.2 | 30.4 ± 1.6 | 3.2 ± 0.5 |
| S40C60 | 4:6 | 700 | 15.3 ± 1.7 | 1.8 ± 0.3 | 14.9 ± 1.1 | 1.7 ± 0.2 |
| | | 800 | 16.8 ± 2.2 | 2.2 ± 0.4 | 13.1 ± 1.9 | 2.1 ± 0.4 |
| | | 900 | 16.0 ± 2.1 | 1.8 ± 0.3 | 15.5 ± 1.9 | 2.9 ± 0.6 |
| | | 1000 | 17.4 ± 1.6 | 2.0 ± 0.2 | 17.5 ± 1.6 | 1.6 ± 0.2 |
| S30C70 | 3:7 | 700 | 10.3 ± 1.7 | 1.6 ± 0.2 | 9.3 ± 1.0 | 1.4 ± 0.2 |
| | | 800 | 11.6 ± 1.7 | 1.1 ± 0.2 | 10.6 ± 1.7 | 1.3 ± 0.3 |
| | | 900 | 11.3 ± 1.7 | 1.1 ± 0.3 | 9.9 ± 1.2 | 0.9 ± 0.1 |
| | | 1000 | 12.3 ± 1.8 | 1.1 ± 0.2 | 10.4 ± 1.7 | 1.2 ± 0.2 |

### Example 2

Effect of Sintering Temperature on Setting Time and Diametral Tensile Strength of the Cements

Tetraethyl orthosilicate (Si(OC₂H₅)₄, TEOS) and calcium nitrate (Ca(NO₃)₂.4H₂O) were used as precursors for SiO₂ and CaO, respectively, and nitric acid as catalyst. Ethanol was used as the solvent. The S70C30, S60C40, S50C50, S40C60, and S30C70 powders were synthesized by sol-gel method and sintered at different temperatures of 800, 900, or 1000° C. for 2 hour. After which, the as-sintered powders were ball-milled using agate jars with agate grinding media in an ethanol medium for 12 hours under Retsch centrifugal ball mill S 100. After drying, in order to prepare the cement, 0.2 g powder was mixed with 0.1 mL of water or 0.5 M Na₂HPO₄. The setting time of the cements was tested using the 400-g Gillmore needle with a diameter of 1 mm according to the international standard ISO 9917-1 for water-based cements (ISO 9917-1, Dentistry-water-based cements part1: powder/liquid acid-base cements. International Standard Organization, 2003). The setting time was recorded when the needle failed to create an indentation of 1 mm in depth in three separate areas. After mixing the cement specimens were placed into a cylindrical stainless steel mould to form the specimen dimension of 6 mm (diameter)×3 mm (height), and stored in an incubator at 100% relative humidity and 37° C. for 1 day. Eight specimens from each group were tested. The diametral tensile testing of cement specimens was conducted on an EZ-Test machine (Shimadzu, Kyoto, Japan) at a loading rate of 0.5 mm/min. The diametral tensile strength (DTS) value of the cement specimens was calculated from the relationship DTS=2P/πbw, where P is the peak load (Newton), b is the diameter (mm) and w is the thickness (mm) of the specimen. The maximal compression load at failure was obtained from the recorded load-deflection curves. At least twenty specimens were used each group. Table 1 presents the results of setting times and DTS values of five calcium silicate cement specimens. When mixed with water, the setting time ranged from 43 to 11 minutes, significantly depending on the used calcium silicate powders. With the increase in the concentration of calcium component, the setting time of the cement became shorter. The cement specimens containing SiO₂/CaO molar ratio ranging from 6:4 to 4:6 had a higher strength than the other two cement specimens. The sintering temperature used for preparation of solid phases did not distinctly affect the setting time and DTS of the cement specimens. On the other hand, 0.5 M Na₂HPO₄ as a liquid phase presented a similar trend to water.

### Example 3

Phase Composition of the Cement Specimens after Mixing with Water

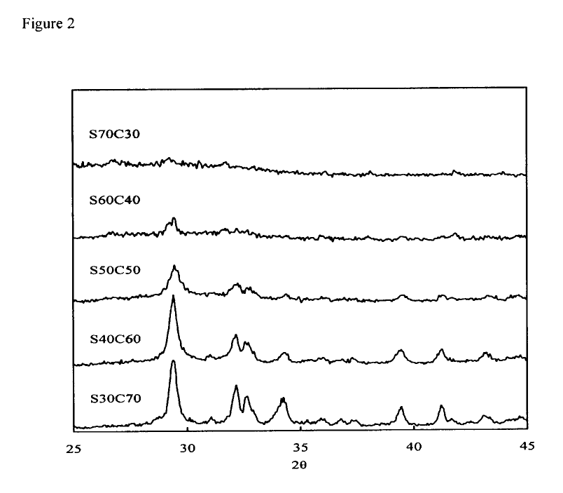

Tetraethyl orthosilicate (Si(OC₂H₅)₄, TEOS) and calcium nitrate (Ca(NO₃)₂.4H₂O) were used as precursors for SiO₂ and CaO, respectively, and nitric acid as catalyst. Ethanol was used as the solvent. The S70C30, S60C40, S50C50, S40C60, and S30C70 powders were synthesized by sol-gel method and sintered at 800° C. for 2 hour, and then were ball-milled using agate jars with agate grinding media in an ethanol medium for 12 hours under Retsch centrifugal ball mill S 100. After drying, in order to prepare the cement, 0.4 g powder was mixed with 0.2 mL of water. The cement was stored in an incubator at 100% relative humidity and 37° C. for 1 day. X-ray diffractometer (XRD) was used to analyze the phase composition of the hardened cement specimens. The products of the hydration process are calcium silicate hydrates (CaO—SiO₂—H₂O, C—S—H) at 29.3° as well as incompletely reacted inorganic component phases as shown in FIG. 2.

### Example 4

Effect of Precursors

Different precursors for SiO₂ and CaO, as listed in Table 2, are used. The precursors for silicon included Tetraethyl orthosilicate (Si(OC₂H₅)₄, TEOS), silicon dioxide (SiO₂), and silicon tetraacetate (Si(CH₃COO)₄). Calcium precursors were calcium nitrate (Ca(NO₃)₂.4H₂O), calcium oxide (CaO), calcium hydroxide (Ca(OH)₂), calcium acetate (Ca(CH₃COO)₂), and calcium chloride (CaCl₂). Nitric acid and ethanol were used as the catalyst and solvent, respectively. Calcium silicate powders with equimolar Si/Ca ratio (S50C50) were synthesized by sol-gel method. The general procedure of sol-gel route, such as hydrolysis and ageing, was adopted. After ageing at 60° C. and solvent vaporization in an oven at 120° C., the as-dried gel was heated in air to 900° C. for holding 2 hour, and then cooled to room temperature to produce various calcium silicate powders. The sintered powders were further ball-milled using agate jars with agate grinding media in an ethanol medium for 12 hours under Retsch centrifugal ball mill S 100. After drying, in order to prepare the cement, the powder was mixed with the (NH₄)₂HPO₄—NH₄H₂PO₄ buffer solution (pH 7.4). The setting time of the cements was tested using the 400-g Gillmore needle with a diameter of 1 mm according to the international standard ISO 9917-1 for water-based cements. In addition, the cement specimens were placed into a cylindrical stainless steel mould to form the specimen dimension of 6 mm (diameter)×3 mm (height), and stored in an incubator at 100% relative humidity and 37° C. for 1 day. The diametral tensile testing of cement specimens was conducted on an EZ-Test machine (Shimadzu, Kyoto, Japan) at a loading rate of 0.5 mm/min. At least ten specimens from each group were tested. Table 2 presents the results of setting times and DTS values of cement specimens. The setting time and DTS values appreciably depended on the type of precursors.

TABLE 2
Setting time and diametral tensile strength (DTS) of the S50C50
cements prepared by mixing with ammonium hydrogen solution

| | TEOS | | SiO₂ | | Si(CH₃COO)₄ | |
|---|---|---|---|---|---|---|
| | Setting | | Setting | | Setting | |
| | time | DTS | time | DTS | time | DTS |
| | (min) | (MPa) | (min) | (MPa) | (min) | (MPa) |
| Ca(NO₃)₂ | 3.7 ± 0.8 | 2.3 ± 0.1 | 12.3 ± 0.8 | 0.4 ± 0.0 | 6.0 ± 1.3 | 2.5 ± 0.1 |
| CaO | 15.2 ± 1.2 | 0.5 ± 0.0 | 20.0 ± 2.7 | 1.7 ± 0.0 | 15.7 ± 2.0 | 1.0 ± 0.0 |
| CaCl₂ | 10.3 ± 1.5 | 0.5 ± 0.1 | 15.5 ± 1.0 | 1.2 ± 0.1 | 12.5 ± 1.4 | 0.7 ± 0.1 |
| Ca(OH)₂ | 32.5 ± 1.9 | 1.9 ± 0.1 | 38.8 ± 2.8 | 0.8 ± 0.1 | 27.2 ± 1.7 | 2.0 ± 0.1 |
| Ca(CH₃COO)₂ | 7.8 ± 0.8 | 1.8 ± 0.1 | 12.5 ± 1.4 | 0.7 ± 0.0 | 13.8 ± 1.0 | 1.8 ± 0.1 |

### Example 5

Phase Composition of the Cement Specimens Containing Gelatin and Chitosan

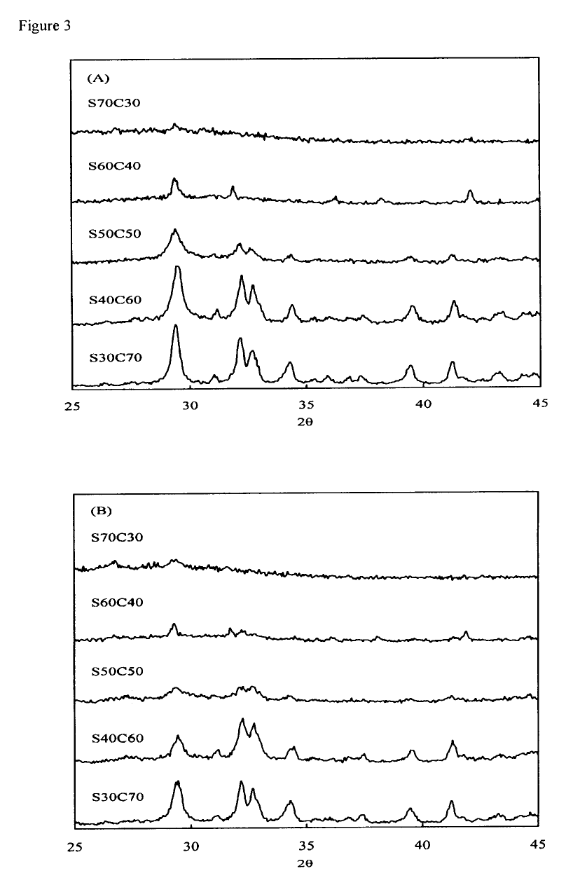

The sol-gel-derived S70C30, S60C40, S50C50, S40C60, and S30C70 powders were sintered at 800° C. for 2 hours. 5 wt % of gelatin was added to the sintered powders. Then, the mixtures with and without gelatin were ball-milled using agate jars with agate grinding media in an ethanol medium for 12 hours under Retsch centrifugal ball mill S 100. After drying, in order to prepare the cement, 0.2 g powder was mixed with 0.1 mL of water or 5 wt % of chitosan solution. Chitosan oligosaccharide lactate powder was dissolved in distilled water at chitosan/(chitosan+water)=5% mass fraction to form the chitosan solution. After mixing, the specimens were stored in an incubator at 100% relative humidity and 37° C. for 1 day. XRD was used to analyze the phase composition of the hardened cement specimens. The product of the hydration process was a calcium silicate hydrate (CaO—SiO₂—H₂O, C—S—H) gel, as shown in FIG. 3. The presence of chitosan in the cement specimen reduced the peak intensity of C—S—H (FIG. 3B).

### Example 6

Effect of Gelatin and Chitosan Contents on Diametral Tensile Strength and Setting Time of the Cements

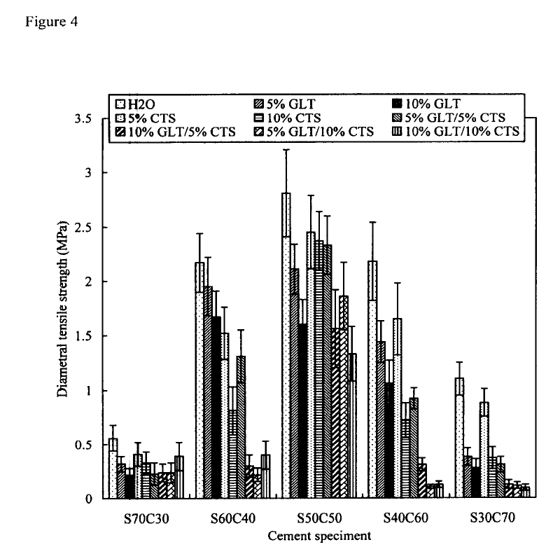

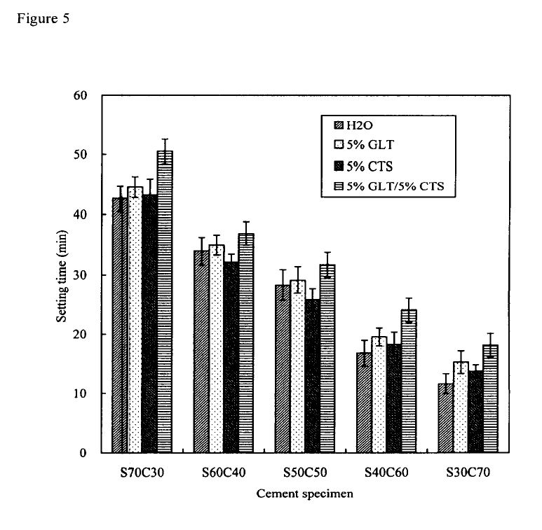

The sol-gel-derived S70C30, S60C40, S50C50, S40C60, and S30C70 powders were sintered at 800° C. for 2 hours. 5 and 10 wt % of gelatin (GLT) was added to the sintered powders. Then, the mixtures with and without gelatin were ball-milled using agate jars with agate grinding media in an ethanol medium for 12 hours under Retsch centrifugal ball mill S 100. After drying, in order to prepare the cement, 0.2 g powder was mixed with 0.1 mL of water or chitosan (CTS) solution. Chitosan oligosaccharide lactate powder was dissolved in distilled water at chitosan/(chitosan+water)=5 or 10% mass fraction to form the chitosan solution. After mixing, the specimens were stored in an incubator at 100% relative humidity and 37° C. for 1 day. The setting time of the cements was tested using the 400-g Gillmore needle with a diameter of 1 mm according to the international standard ISO 9917-1 for water-based cements. The setting time was recorded when the needle failed to create an indentation of 1 mm in depth in three separate areas. In addition, the cement specimens were placed into a cylindrical stainless steel mould to form the specimen dimension of 6 mm (diameter)×3 mm (height), and stored in an incubator at 100% relative humidity and 37° C. for 1 day. Eight specimens from each group were tested. The diametral tensile testing of cement specimens was conducted on an EZ-Test machine (Shimadzu, Kyoto, Japan) at a loading rate of 0.5 mm/min. At least twenty specimens were used each group. FIG. 4 shows diametral tensile strength (DTS) of various cement specimens. The addition of gelatin or chitosan could decrease DTS of the cement specimens. Similarly, they might also affect the setting time as shown in FIG. 5.

### Example 7

Injectability of the Cement

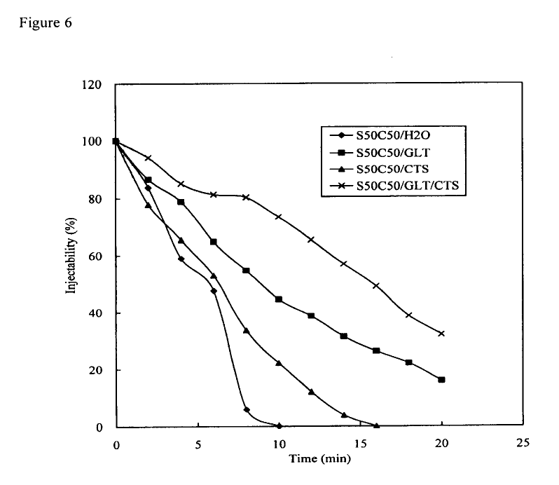

The sol-gel-derived S50C50 powders were sintered at 800° C. for 2 hours. 5 wt % of gelatin (GLT) was added to the sintered powders. Then, the mixtures with and without gelatin were ball-milled using agate jars with agate grinding media in an ethanol medium for 12 hours under Retsch centrifugal ball mill S 100. After drying, in order to prepare the cement, the powder was mixed with water or 5 wt % of chitosan (CTS) solution liquid-to-powder (L/P) ratio of 0.6 mL/g. The injectability was evaluated by extruding the paste through a disposable syringe of 5-mL equipped with the needle having an opening of 2.0 mm. The appropriate amount of cement paste was used, and “injectability” was taken to mean the percentage by weight of that part of this amount of paste, which could be extruded from such syringes by hand. The injectability of the cement pastes was determined 2 min after the beginning of mixing powder and liquid. FIG. 6 shows the injectability of the S50C50 cement with and without gelatin and chitosan. Both gelatin and chitosan can improve the injectabilty of the cement specimens.

### Example 8

Diametral Tensile Strength and Morphology for Various Cement Specimens after Immersion in Simulated Body Fluid

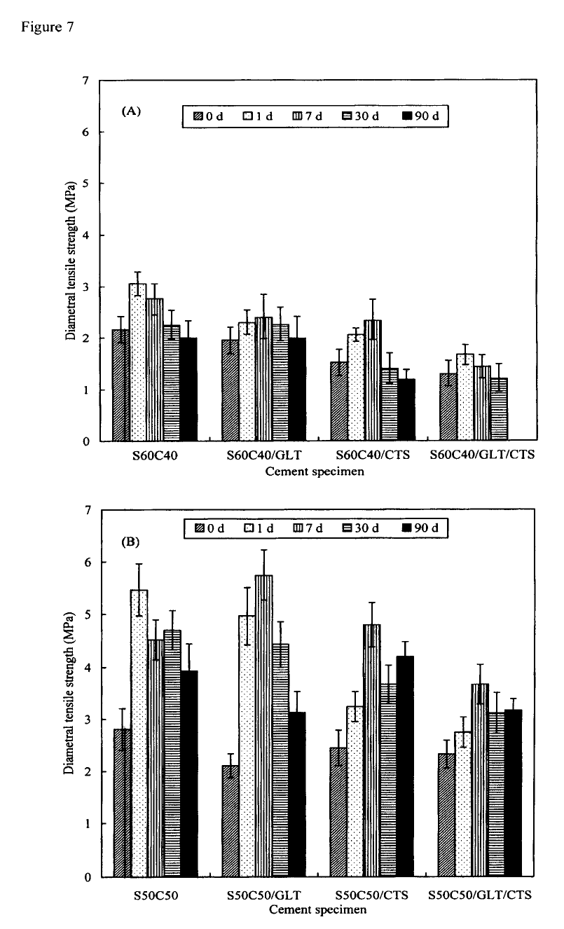

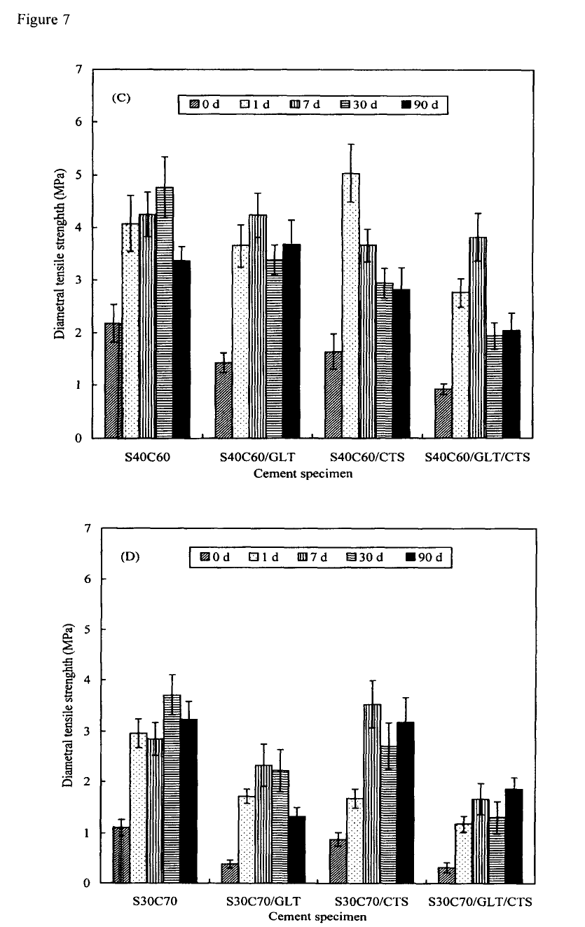

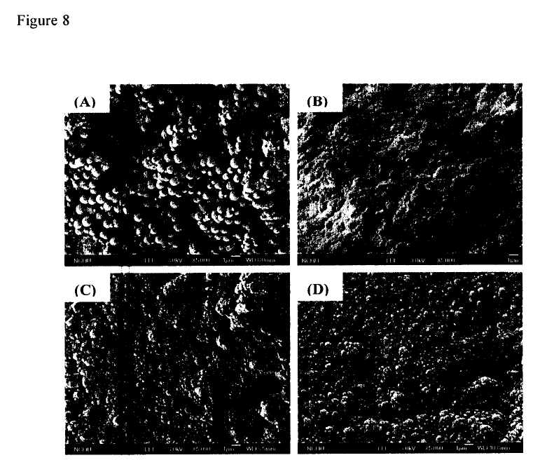

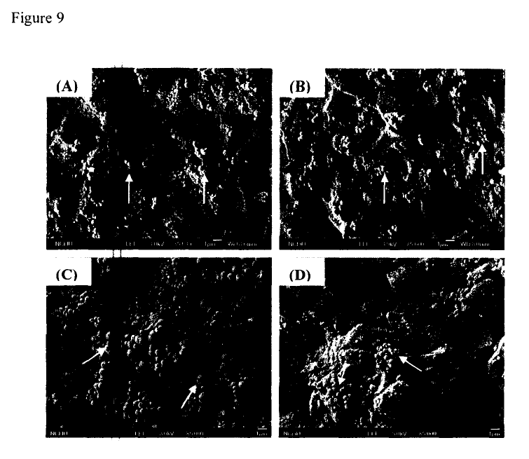

The sol-gel-derived S60C40, S50C50, S40C60, and S30C70 powders were sintered at 800° C. for 2 hours. 5 wt % of gelatin (GLT) was added to the sintered powders. Then, the mixtures with and without gelatin were ball-milled using agate jars with agate grinding media in an ethanol medium for 12 hours under Retsch centrifugal ball mill S 100. After mixing with water or 5 wt % chitosan (CTS) solution, each hardened specimen that was stored in an incubator at 100% relative humidity and 37° C. for one day was immersed in physiological solution for the predetermined periods of time at 37° C. to evaluate the cement bioactivity. A simulated body fluid (SBF), which ionic composition is similar to that of human blood plasma, was used for immersion tests. The SBF solution consisted of 7.9949 g NaCl, 0.3528 g NaHCO₃, 0.2235 g KCl, 0.147 g K₂HPO₄, 0.305 g MgCl₂.6H₂O, 0.2775 g CaCl₂, 0.071 g Na₂SO_{distilled H}₂O and was buffered to pH 7.4 with hydrochloric acid (HCl) and trishydroxymethyl aminomethane (CH₂OH)₃CNH₂). The solution in a shaker water bath was not changed daily. After immersion in SBF for different periods of time, the specimens were removed from the vials and performed the tensile testing using an EZ-Test machine. At least twelve specimens from each group were tested. DTS of almost immersed cement specimens had a greater value than the hardened cements (FIG. 7). After immersion, the specimens were removed from the vials to observe morphologies using a SEM. FIGS. 8 and 9 indicate that the cement specimens induce the formation of apatite spherulites, indicating the bioactivity.

### Example 9

Cell Viability and Morphology

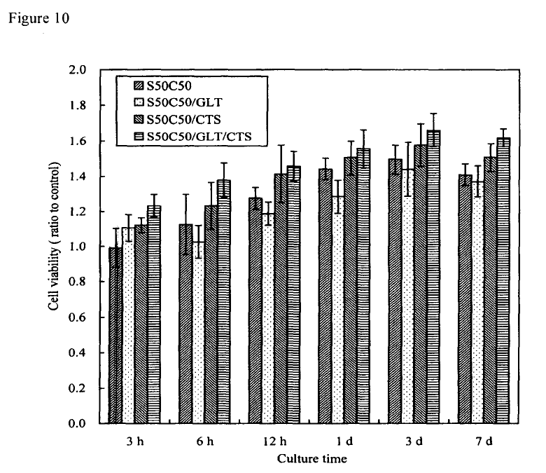

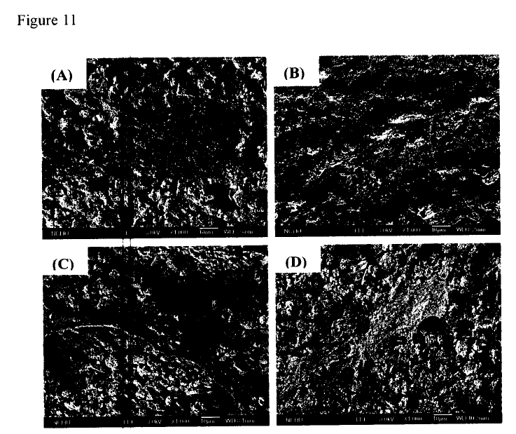

Cement biocompatibility was evaluated by incubation with human bone marrow-derived mesenchymal stem cells (hMSCs) obtained from Osiris (Worthington Biochemical, Lakewood, N.J.) at passage 4. The sol-gel-derived S50C50 powders were sintered at 800° C. for 2 hours. 5 wt % of gelatin (GLT) was added to the sintered powders and ground for 12 hours. After drying, in order to prepare the cement, the powder was mixed with water or 5 wt % of chitosan (CTS) solution liquid-to-powder (L/P) ratio of 0.5 mL/g. The cement specimens were stored in an incubator at 100% relative humidity and 37° C. for one day. For cell culture, the 1-day setting cement discs were sterilized by soaking in 75% ethanol and exposure to ultraviolet (UV) light for 2 hour. The hMSC cells were seeded to sterilized S50C50 cement at a density of 5×10³ cells/well in a 24-well plate. The cells were grown in the osteogenic induction medium consisted of Dulbecco's modified Eagle's medium (DMEM) supplemented with 15% fetal bovine serum (FBS), 584 mg/L glutamine, 0.1 mM MEM sodium pyruvate, 100 U/mL penicillin, 100 μg/mL streptomycin, 50 mg/L ascorbic acid, 0.1 mM MEM nonessential amino acids, and 100 nM dexamethasone and 10 mM β-glycerophosphate disodium salt hydrate. Cultures were incubated at 37° C. in a 5% CO₂ atmosphere for different time periods. The medium was changed every three days. The Cell Proliferation Reagent WST-1 (Roche Diagnostics, Mannheim, Germany) was used to assess cell attachment and proliferation, which is based on the cleavage of a tetrazolium salt (WST-1) by the mitochondrial dehydrogenase of living cells. Briefly, three hours before the end of incubation, 100 uL of WST-1 solution (Sigma) and 900 uL of medium were added to each well. 200 μL of the solution in each well was transferred to a 96-well tissue culture plate. Plates were read in a microtiter plate reader (Bio-Rad Benchmark Plus™ Microplate Spectrophotometer) at 440 nm with a reference wavelength of 650 nm. Sample analysis results were obtained in triplicate from four separate experiments. hMSCs cultured on tissue culture plates (TCPs) were used as a control. In FIG. 10 WST-1 assay shows that the number of viable cells increases with an increased incubation, indicating a good biocompatibility. The chitosan-containing cement specimens show a better biocompatibility compared with those specimens without chitosan. Cell morphology on the cement surfaces was examined by scanning electron microscopy (SEM). The specimens were washed by phosphate buffer solution three times and fixed with 2.5% glutaraldehyde at 4° C. for 2 hours. Then the cements were dehydrated in a graded ethanol series for 20 minutes at each concentration and dried over night. The dried specimens were mounted on stubs, coated with gold layer. The cell morphology on the cement surface was observed using JEOL JSM-6700F SEM. The evaluation of SEM images confirmed that cells appeared to have firmly anchored on the cement surfaces (FIG. 11).

What is claimed is:

1. A method for producing polymer-containing calcium silicate bone cement, comprising
(a) mixing calcium salt with silicon compound;
(b) treating the mixture of step (a) with a sol-gel process;
(c) heating the mixture of step (b);
(d) adding materials for enhancing plasticity to the mixture of step (c);
(e) grinding the mixture of step (d) into powder; and
(f) adding the powder to water or phosphate solution with or without the materials for enhancing plasticity,
wherein the materials for enhancing plasticity are selected from the group consisting of the polymeric and oligomeric materials with the functional group —NH₂, —OH, —CO or —CH₃.

2. The method of claim 1, wherein the calcium salt is calcium nitrate, calcium oxide, calcium hydroxide, calcium acetate, calcium carbonate or calcium chloride.

3. The method of claim 1, wherein the silicon compound is calcium silicohydride, silicon dioxide or silicon tetraacetate.

4. The method of claim 3, wherein the silicohydride has the following formula: 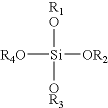
wherein R₁, R₂, R₃ or R₄ is C₁₋₆ alkyl.

5. The method of claim 4, wherein R₁, R₂, R₃ or R₄ is C₂H₅.

6. The method of claim 1, wherein the mixture has a molar ratio of calcium to silicon ranging from 10 to 0.1.

7. The method of claim 6, wherein the molar ratio of calcium to silicon ranging from 4 to 0.25.

8. The method of claim 1, wherein the polymeric and oligomeric materials as solid phases or liquid phases are selected from the group consisting of gelatin, collagen, chitosan, chitin, cellulose, alginate, hyaluronic acid, poly(lactic acid), poly(glycolic acid), and poly(lactic-co-glycolic acid).

9. The method of claim 1, wherein the materials for enhancing plasticity are weighed in the range of 2-30 total wt %.

10. The method of claim 1, wherein the sol-gel process comprises following steps:
(a) mixing the mixture with a diluted solvent selected from the group consisting of nitric acid and ethanol for 1˜12 hours;
(b) placing the mixture at the temperature of 20˜100° C. for 1˜7 days; and
(c) drying the mixture at −40˜150° C.

11. The method of claim 1, wherein the heating process comprises following steps:
(a) heating the dried mixture to 700˜1300° C. at the rate of 1˜40° C./min;
(b) maintaining the mixture at a constant temperature ranges from 700˜1300° C.; and
(c) cooling the mixture to room temperature by air-cooling, water-cooling or fast cooling techniques to obtain calcium silicate powder.

12. The method of claim 1, wherein the grinding process comprises following steps:
(a) mixing the calcium silicate powder containing the materials for enhancing plasticity with alcohol;
(b) grinding the powder with a ball bill for 0.5˜3 days; and
(c) drying the powder at −40˜60° C.

13. The method of claim 1, wherein the powder to water or phosphate solution is at a ratio of 1 g/0.3-2 mL.

14. The method of claim 13, wherein the powder to water or phosphate solution is at a ratio of 1 g/0.4-0.8 mL.

15. The method of claim 1, wherein the phosphate solution contains an anion selected from phosphate radical (PO₄³⁻), monohydric phosphate radical (HPO₄²⁻) or dihydric phosphate radical (H₂PO₄⁻)

16. The method of claim 1, wherein the phosphate solution contains a cation selected from ammonium or a member of group I A.

17. The method of claim 15, wherein the anion is in the concentration of 0.12˜5 M.

18. A composition for polymer-containing calcium silicate bone cement, comprising
(a) calcium salt;
(b) silicon compound;
(c) materials for enhancing plasticity; and
(d) pharmacological acceptable solution with or without the materials for enhancing plasticity,
wherein the materials for enhancing plasticity are selected from the group consisting of the polymeric and oligomeric materials with the functional group —NH₂, —OH, —CO or —CH₃.

19. The composition of claim 18, wherein the pharmacological acceptable solution is water, sodium chloride solution, calcium chloride solution or phosphate solution.

20. The composition of claim 18, which is applied to orthopedic surgery, medical or dental application.

21. The composition of claim 18, which is applied to replacement bone or tooth material.

22. The composition of claim 18, which further comprises an excipient for oral administration.
